(19) United States
(12) Patent Application Publication
(10) Pub. No.: US 2016/0272666 A1
(43) Pub. Date: Sep. 22, 2016

(54) Partially Crystalline Glucamide Compositions And Method For Preparing Same

(71) Applicant: CLARIANT INTERNATIONAL LTD., Muttenz (CH)

(72) Inventors: Peter KLUG, Grossostheim (DE); Gerd DAHMS, Duisburg (DE)

(73) Assignee: Clariant International Ltd., Muttenz (CH)

(21) Appl. No.: 15/037,172

(22) PCT Filed: Nov. 18, 2014
(86) PCT No.: PCT/EP2014/003081

§ 371 Date: May 17, 2016

(30) Foreign Application Priority Data
Nov. 20, 2013 (EP) 13193711.2

(51) Int. Cl. C07H 5/06; A61Q 19/10; C11D 17/00; A61Q 5/02; C11D 1/66; A61K 8/60; C07H 1/00

(52) U.S. Cl. CPC C07H 5/06; A61K 8/60; A61Q 19/10; C07H 1/00; A61Q 5/02; C11D 1/662; C11D 17/0026; C07B 2200/13; A61K 2800/5922

(57) ABSTRACT

The invention relates to a partially crystalline N-alkyl-N-acylglucamine composition comprising at least two different acylglucamines with acyl groups selected from among saturated or unsaturated C₆-C₂₂ acyls, the composition having a partially crystalline structure at room temperature with at least one significant X-ray reflection in each of the 2theta ranges >=13.5° to <=15.1° and >=15.5° to <=17.4°, and the X-ray reflections being obtained by a powder diffraction diffractogram in Bragg-Brentano geometry.

The invention relates to a partially crystalline N-alkyl-N-acylglucamine composition comprising at least two different acylglucamines having acyl groups selected from the group of saturated or unsaturated C₆-C₂₂ acyls, wherein the composition at room temperature has a partially crystalline structure with at least one significant X-ray reflection in each of the 2Theta ranges ≧13.5° to ≦15.1° and ≧15.5° to ≦17.4°, wherein the X-ray reflections are obtained by a powder diffraction diffractogram in Bragg-Brentano geometry.

The quality requirements of modern laundry detergents and cleaning agents have become markedly more complex in recent decades. In addition to a good cleaning efficiency, the consumer currently expects a good (environmental) compatibility, tailor-made handleability, ready biodegradability and also the use of renewable resources. A substantial point for meeting this profile of requirements lies here in the provision of detergent substances, in most cases amphiphilic molecules, which in the context of the chosen formulation and application situation are to exhibit very good properties in important handling parameters such as cold solubility behavior, dissolution behavior, flow behavior and dilution behavior, and also soil-suspending ability and ability to thicken. This conflicting situation is, precisely in the field of detergent substances, a great challenge, since the specific mode of action of amphiphilic substances, in contrast to the interactions of other classes of substances, in most cases, may not adequately be described by simple individual molecule—individual molecule relationships. Generally, the most familiar industrial fields of application of these substances, such as, for example, antifoams, (W/O)/(O/W)-emulsifiers, wetting agents, surfactants or solubilizers, are based not only on a complex interaction of the amphiphiles with one another but also on a related additional interaction of the amphiphiles with the potential target matrix. The understanding of both classes of interaction, the amphiphile-amphiphile and amphiphile-target matrix, is in this case of equal importance in order to be able to provide tailor-made properties for the respective usage situation.

In the substance class of nonionic surfactants, N-alkyl-N-acylglucamines (glucamides) have proved to be particularly suitable to be able to meet the above requirement profile. This substance class is dermatologically highly acceptable, readily biologically degradable, exhibits a markedly good cleaning action and may be prepared from renewable raw materials. For this reason, in the patent literature there is a multiplicity of documents which cite differing strategies for preparing these molecules.

Thus, for example, U.S. Pat. No. 5,194,639 A describes a method for preparing purified, substantially non-cyclic polyhydroxy fatty acid amides, in which a fatty acid ester and an N-alkylpolyhydroxy amine react with one or more hydroxylated or alkoxylated solvents in the presence of a basic catalyst. Said process has the following parameters: a) the basic catalyst is an alkoxide, b) the process is carried out in a temperature range from approximately 25° C.-130° C., the weight ratio between fatty acid ester and N-alkylpolyhydroxyamine is at least approximately 1:1, said fatty acid ester is a C₁₂-C₂₀ fatty acid ester and e) said solvent is a C₁-C₄ alcohol, ethylene glycol, propylene glycol, glycerol, alkoxylated alcohol or mixtures of same.

A further pathway for preparing linear glucamide surfactants can be found in U.S. Pat. No. 5,338,486. Therein, the glucamide is obtained by reaction of an N-alkylglucamine, for example N-methylglucamine, of a fatty acid ester, for example coconut methyl ester, in the presence of a highly concentrated catalyst selected from trilithium phosphate, trisodium phosphate, tripotassium phosphate, tetrasodium pyrophosphate, tetrapotassium pyrophosphate, pentasodium tripolyphosphate, pentapotassium tripolyphosphate, lithium carbonate, sodium carbonate, potassium carbonate, disodium tartrate, dipotassium tartrate, sodium potassium tartrate, trisodium citrate, tripotassium citrate, basic sodium silicates, basic potassium silicates, basic barium aluminosilicates, basic potassium aluminosilicates and mixtures thereof.

Furthermore, in U.S. Pat. No. 5,380,891 A there is a possible embodiment for synthesizing linear N-alkylglucamides. Therein, an N-alkylglucamine, for example N-methylglucamine, a fatty acid ester, for example coconut oil, is reacted in the presence of a phase-transfer agent, a nonionic surface-active material, preferably an already pre-prepared product of the process, preferably in the presence of an alkaline catalyst.

In U.S. Pat. No. 5,723,673 A, in contrast, a pathway for synthesizing N-alkylpolyhydroxy amine amides with good color is described. An N-alkylpolyhydroxyalkylamine, such as N-methylglucamine, having a Gardner color number less than 1 is reacted with a source of fatty acid acyl groups such as methyl esters, anhydrides and/or fatty acids having a transmission of greater than 98% at 460 nm in organic hydroxy solutions such as methanol. The N-alkylpolyhydroxyamines can be purified by crystallization and/or by bleaching to obtain an improved color. To reduce the fraction of cyclic reaction products, the reaction can preferably be carried out at low temperatures, in short time periods and at a low catalyst fraction. The resultant amide product can, in addition, be purified by means of anion- and cation-exchange columns, with removal of soaps and amines.

WO 9 410 130 A1, on the other hand, describes the preparation of N-alkanoylpolyhydroxyalkylamines Z—NR¹—CO—R², wherein Z is the polyhydroxyalkyl group of a mono- or oligosaccharide, R¹ is hydrogen or C₁-C₈ alkyl and R² is C₁-C₂₁ alkyl. The preparation proceeds by reacting polyhydroxyalkylamines Z—NH—R¹ with carboxylic acid alkyl esters R³—O—CO—R², wherein R³ is a C₁- to C₄-alkyl group, in the presence of a basic catalyst. For this purpose (a) the total amount of the ester is charged first, heated to the reaction temperature, and the amine is added in the form of a melt, with progression of the reaction, wherein alcohol R³OH formed is continuously distilled off, (b) the reaction is carried out at a temperature from 70 to 110° C. and (c) the reaction is performed in the absence of organic solvents.

It has emerged, however, that the processing and detergent properties of the N-alkyl-N-acylglucamines are particularly advantageous when these substances are present in an organized manner in solution or subcooled melt within ordered structures. This may be observed, in particular, when a partially crystalline state of the glucamides is formed. This partially crystalline state results from the specific interactions of the glucamides among one another which may be attributed to the geometries of the molecules themselves and, obviously, accordingly also to the byproduct profile of the process pathway chosen. The partially crystalline structures discussed here as preferred apparently demonstrate a complex arrangement which is not ascribable to simple micellar geometries. Partially crystalline states of N-alkyl-N-acylglucamines may not be obtained by the methods mentioned in the prior art and are, in particular, also not discussed.

It is to this extent the object of the present invention to provide compositions having N-alkyl-N-acylglucamines in an ordered partially crystalline structure. In addition, it is the object of the present invention to specify an advantageous method for preparing these partially crystalline N-alkyl-N-acylglucamine compositions.

The invention accordingly relates to a partially crystalline N-alkyl-N-acylglucamine composition comprising at least two different acylglucamines having acyl groups selected from the group of saturated or unsaturated C₆-C₂₂ acyls, wherein the composition at room temperature has a partially crystalline structure with at least one significant X-ray reflection in each of the 2Theta ranges ≧13.5° to ≦15.1° and ≧15.5° to ≦17.4°, wherein the X-ray reflections are obtained by a powder diffraction diffractogram in Bragg-Brentano geometry. Surprisingly, it has become apparent that N-alkyl-N-acylglucamines that have different C chain sections and in addition are present in the form of solutions or mixtures also form defined partially crystalline structures. This is in contrast to N-alkyl-N-acylglucamine monocrystals which are composed of only one defined N-alkyl-N-acylglucamine species and for which, owing to the structural regularity of the individual glucamide building block, a regular arrangement would be expected. Without wishing to be bound by theory, these partially crystalline ordered structures, despite different chain lengths in solution or in a mixture, can also result when the further N-alkyl-N-acylglucamine byproduct spectrum is ignorable in such a manner that a sufficiently high number of molecules is ready to depress the free energy of the system by forming ordered structures. In particular, for example cyclic (by)products can cause marked interference of packing geometries to be expected, which overall would counteract a partially crystalline arrangement. Further surprisingly, it has become apparent that this structure to a great extent reacts tolerantly to further additives, without leaving the partially crystalline state. A number of application advantages can hereby result for the composition. The ready dilutability by addition of solvents has already been mentioned above. Furthermore, an improved soil suspension ability can also be established, since soil particles can be incorporated more reliably into larger partially crystalline structures. This can contribute to a generally improved detergent action of a partially crystalline composition of amphiphilic molecules.

A partially crystalline structure in the meaning of the invention is present when the composition within a recording of a powder diffraction diffractogram has at least two defined reflections in the above-cited 2Theta angle ranges. This is in contrast to noncrystalline compositions which do not have ordered structures in the composition, and so, in a PXRD image, only one amorphous halo without defined peaks is found. To determine whether, in the stated angle range, a significant reflection is present, the criterion of relative intensity is used. For this purpose, the relation between the reflection intensity in the angle range considered and the reflection intensity of the strongest reflection in the range between 3° and 40° (2Theta) is considered. Only when this relation is above a certain threshold value, for example 20% (relative to the strongest reflection), is a significant reflection present in the angle range under consideration. This mode of consideration takes into account the use of only arbitrary intensities (counts/sec). The size of the claimed 2Theta ranges results from the method-related presence of textural effects. The minimum intensity for the presence of a significant reflection for the 2Theta ranges cited is stated in the examples for the respective structural type.

Room temperature for the purposes of the invention is within a temperature range between 20 and 25° C.

The improved properties of the partially crystalline compositions may be achieved, in particular, in the case of compositions having ordered structures that give significant reflections in the above-specified 2Theta ranges. The 2Theta ranges ≧13.5° to ≦15.1° and ≧15.5° to ≦17.4° indicate periodic structures having mean size ranges of 6.1 Å (first range) and 5.3 Å (second range). Without wishing to be bound by theory, these periodic structures can be of importance in particular in the case of swelling and rearrangement processes of the crystalline structures and in this manner contribute to improved detergent and handling properties.

In particular, in a further embodiment of the invention, the diffractograms of the composition can have a further significant reflection which corresponds to a cell axial dimension of greater than or equal to 30 Å and less than or equal to 100 Å. This reflection shows here at least an intensity of greater than or equal to 20% in relation to the intensity of the strongest reflection in the range between 3° and 40° (2Theta).

A N-alkyl-N-acylglucamine composition in the context of the invention can consist of N-alkyl-N-acylglucamines of different chain lengths or contain them. Additional ingredients of the compositions can comprise further substances, for example, which are described in the prior art as detergent or nondetergent substances that are usable in surfactant systems. This group can generally include solvents, builders, bleachers, softeners, enzymes, fragrances or suspending agents. It has surprisingly been found that the partially crystalline composition according to the invention reacts in a very tolerant manner to the incorporation of these ingredients, and these substances may be substantially integrated without loss of the partially crystalline structure. Particular preference, in contrast, is given to a composition in which the detergent substances of the composition only consist of N-alkyl-N-acylglucamines.

The N-alkyl-N-acylglucamines conform in this case to the following formula:

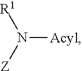

wherein Z is a sugar group or generally a polyhydroxyalkyl group, R¹ is a C₁-C₄ alkyl group and acyl is a C₆-C₂₂ acyl group of the structure

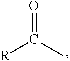

wherein R is a C₅-C₂₁ saturated or monounsaturated or polyunsaturated alkyl group.

The composition has according to the invention at least two different acylglucamines having acyl groups selected from the group of saturated or unsaturated C₆-C₂₂ acyls. The composition can have here between at least 2 and 10, preferably between 2 and 9, and in addition preferably between 2 and 8 different N-alkyl-N-acylglucamines which differ in their acyl chain length. Despite this number of different acyl chain lengths, it has been found that the composition according to the invention is able to form an at least partially crystalline structure which has periodic structures in the above-cited ranges and accordingly can contribute to improved detergent properties. For further simplification, in the sections hereinafter, the nomenclature CX glucamides is used, wherein, for example, the abbreviation C₁₂ glucamide is an N-alkyl-N-acylglucamine having an acyl chain length of 12 carbon atoms.

The compositions according to the invention exhibit here at least one significant X-ray reflection in each of the specified 2Theta ranges. This means that the composition has at least one periodic/partially crystalline structure which corresponds to an order of size corresponding to the angle range. A significant X-ray reflection is present when the intensity of the reflection maximum, compared with the maximum intensity of the strongest signal of the range, exceeds a certain threshold value. These percentage threshold values for the classification of the individual X-ray reflections are range-dependent and are cited in the examples. The exact experimental conditions for determining the reflection positions in the PXRD images are likewise listed in the context of the examples. The conversion of the angle range to the size range is known to those skilled in the art here. In addition, in the cited angle ranges, however, more than one reflection can also occur. This would then indicate partially crystalline structures with slightly different orders of size in this range. This modification is also intended to be included in the context of the present invention.

The reflection positions of the composition according to the invention are determined by means of recording a powder diffraction diffractogram in Bragg-Brentano geometry. This geometry is known to those skilled in the art for recording X-ray powder spectra and has proved to be particularly suitable for reliable determination of the reflection positions of partially crystalline compositions.

In an additional embodiment according to the invention, the composition at room temperature can have at least one further significant X-ray reflection in a 2Theta range from ≧7.5° to ≦8.5°. Partially crystalline compositions which, in addition to the abovementioned X-ray reflections, still have at least one further reflection around approximately 8° (2Theta) have proved to be particularly efficient in the field of the preparation and in the use properties. Without wishing to be bound by theory, a partially crystalline composition having these three reflection positions can result, in particular, when very few glucamide byproducts destroying symmetry, and a sufficient fraction of C₁₂ acylglucamines, are present in the composition. This partially crystalline structure has proved to be particularly stable and flexible for the inclusion of further non-glucamides that also destroy symmetry. In addition, this partially crystalline composition can readily be diluted down with further solvents, while retaining the structure. To obtain this preferred partially crystalline structure, the fraction of the C₁₂ acylglucamides of the entire glucamide composition in addition can advantageously be greater than or equal to 30 mol %, and less than or equal to 85 mol %, preferably greater than or equal to 35 mol % and less than or equal to 80 mol %, and further preferably greater than or equal to 40 mol % and less than or equal to 75 mol %.

In a further embodiment, partially crystalline compositions can be particularly suitable which, in addition to the additional significant reflection in the range between ≧7.5° to ≦8.5°, still have at least one further significant reflection in each of the 2Theta ranges between 5.6° and 6.5°, and between ≧11.6° and ≦12.6°. Compositions having this partially crystalline structure defined by five reflection positions can be distinguished by a very good detergent action and good handleability.

In a preferred embodiment variant, the composition at room temperature can have at least one further significant X-ray reflection in a 2Theta range from ≧6.2° to ≦7.5°. A further subquantity of the N-alkyl-N-acylglucamines claimed according to the invention can exhibit particularly good application properties if, in addition to the significant reflections at ≧13.5° to ≦15.1° and ≧15.5° to ≦17.4° (2Theta), at least one further significant reflection is detectable between ≧6.2° and ≦7.5°. Without wishing to be bound by theory, these advantageous compositions can result in cases in which the fraction of the C₁₂ glucamides is small in the context of the compositions. These compositions can exhibit in particular very stable foam properties and can advantageously contain greater than or equal to 0 mol % and less than or equal to 3 mol %, preferably greater than or equal to 0 mol % and less than or equal to 2 mol %, and further preferably greater than or equal to 0 mol % and less than or equal to 1 mol % C₁₂ glucamides.

In an alternative embodiment variant, the partially crystalline composition can comprise C₁₂ N-methyl-N-acylglucamine and the C₁₂ acylglucamine fraction, based on the total N-methyl-N-acylglucamine content, can be greater than or equal to 25 mol % and less than or equal to 95 mol %. Within this embodiment, the composition consists of a plurality of N-alkyl-N-acylglucamines, wherein the fraction of the C₁₂-methyl-glucamide chain section is in the abovementioned range. Compositions having a C₁₂-acyl-methyl-glucamine content in these quantitative ranges in this case have proved to be particularly balanced in the context of detergent action, solubility and foam behavior. This is very probably caused by a particularly favorable mean chain length of the total chain section of the composition. To obtain this preferred partially crystalline structure, the fraction of the C₁₂-acyl-methyl-glucamides of the overall composition, in addition, advantageously can be greater than or equal to 30 mol % and less than or equal to 90 mol %, preferably greater than or equal to 35 mol % and less than or equal to 85 mol %, and further preferably greater than or equal to 40 mol % and less than or equal to 80 mol %.

A further preferred embodiment relates to a composition which additionally comprises a solvent selected from the group of mono-, di- or trihydric alcohols. This group of solvents has proved to be particularly expedient for diluting the compositions according to the invention, wherein these solvents can be added in wide concentration ranges without losing the partially crystalline structure of the N-alkyl-N-acylglucamines. Without wishing to be bound by theory, these mono-ols or polyols can be incorporated in a manner favorable to symmetry between the very probably associated, hydrophilic sugar groups of the N-alkyl-N-acylglucamines, in such a manner that only a slight expansion of the observable crystalline structures may occur. For example greater than or equal to 2.5% by weight and less than or equal to 30% by weight, preferably greater than or equal to 5% by weight and less than or equal to 25% by weight, and further preferably greater than or equal to 10% by weight and less than or equal to 20% by weight may be incorporated from this group of solvents. In the context of a particular embodiment, the solvent can either consist of glycerol or propylene glycol or mixtures thereof, or contain them. This has proved to be particularly expedient in relation to the flowability of the composition.

In a further embodiment of the invention, the concentration of the N-alkyl-N-acylglucamines in the composition can be greater than or equal to 5% by weight and less than or equal to 65% by weight. This fraction of N-alkyl-N-acylglucamines has proved particularly advantageous for forming at least partially crystalline structures. The interactions of the compositions according to the invention are in this case apparently sufficient in such a manner that even larger amounts of non N-alkyl-N-acylglucamines do not lead to a great interference in the ordered structures. In particular, the concentration of the N-alkyl-N-acylglucamines in the composition, in addition, can be greater than or equal to 10% by weight, and less than or equal to 60% by weight, preferably greater than or equal to 15% by weight, and less than or equal to 50% by weight, and further preferably greater than or equal to 25% by weight, and less than or equal to 55% by weight. These concentration ranges permit, inter a/ia, effective logistics in the preparation chain. In the context of low N-alkyl-N-acylglucamine concentrations, it may also be useful to carry out the PXRD studies by means of capillaries. In the event of low signal strengths, this can improve the statistics of the measurements.

In a further characteristic, the fraction of at least one N-alkyl-N-acylglucamine in the composition, based on the total N-alkyl-N-acylglucamine content, can be greater than or equal to 40 mol %, and less than or equal to 95 mol %. Surprisingly, it has been found that the composition, to form an at least partially crystalline structure, need not necessarily consist of only one N-alkyl-N-acylglucamine species. This is surprising, since regular structures form more easily when the symmetry of the individual building blocks is the same. Interferences in the symmetry, e.g. due to different acyl chain lengths, should lead to a different spatial requirement of the chains, which should first of all counteract a partially crystalline arrangement. A particularly expedient incorporation of the N-alkyl-N-acylglucamines, and, as a consequence thereof, also particularly expedient application properties, have resulted in the above-stated relative composition. Further preferred embodiments can also result when the fraction of at least one N-alkyl-N-acylglucamine in the composition, based on the total N-alkyl-N-acylglucamine content, is greater than or equal to 45 mol % and less than or equal to 85 mol %, preferably greater than or equal to 45 mol % and less than or equal to 75 mol %. These relationships can result in a composition having an at least partially crystalline structure and permit an adaptation of the individual products to the application properties desired respectively.

In addition, the invention relates to a method for preparing a partially crystalline N-alkyl-N-acylglucamine composition comprising the steps:
a) bringing the N-alkylglucamine to reaction with a base in aqueous solution,
b) drying the reaction product of step a),
c) initially charging at least two carboxylic acid alkyl esters having a different C₆-C₂₂ chain length,
d) adding in portions the dried reaction product obtained under b) to the mixture of c) and
e) reacting to completion the composition of d) under reduced pressure.

This method of preparation has turned out to be particularly efficient and low in byproducts, in such a manner that, by this process, N-alkyl-N-acylglucamines may be synthesized which are particularly suitable for forming at least partially crystalline structures. Without wishing to be bound by theory, by combining steps a) and b), a basic N-alkylglucamine can be obtained which very probably has the center of the basic properties at the nitrogen. This basic nitrogen can, in particular, be suitable for highly selective reaction with the carboxylic acid alkyl esters under basic cleavage of same. By this method procedure, apparently the formation of unwanted, sterically different, for example cyclic, byproducts can be suppressed to the extent that products that are sterically very similar having an increased ability for forming partially crystalline structures result. This can be advantageous for obtaining improved application properties. The fraction of unwanted byproducts and unreacted starting material is also mentioned as a disadvantage of the known methods by other patent documents, for example WO9410130 A1.

To synthesize the N-alkyl-N-acylglucamines, as N-alkylglucamines, in particular N-alkylglucamines having an alkyl group R¹ selected from the group of the C₁-C₄ alkyls can be used. These alkyl groups comprise, in particular, methyl, ethyl, n-propyl, isopropyl, n-butyl, isobutyl, sec-butyl, tert-butyl groups. To obtain at least partially crystalline compositions, in this case, preferably methyl, ethyl, n-propyl and n-butyl can be used. These N-alkyl groups can lead to N-alkyl-N-acylglucamines that are particularly packaging friendly. Particularly application-friendly N-alkyl-N-acylglucamines can, in addition, result via the use of N-methylglucamines. For this reason, the use of N-methylglucamine can be preferred.

The polyhydroxyalkyl group Z of the N-alkylglucamines can be derived from monosaccharides such as erythrose, threose, ribose, arabinose, xylose, lyxose, allose, altrose, glucose, mannose, gulose, idose, galactose, talose or fructose, or derivatives thereof such as glucuronic acid or deoxyribose, or from oligosaccharides, in particular from disaccharides such as sucrose, lactose, trehalose, maltose, cellobiose or gentiobiose, in addition, also from trisaccharides such as raffinose. In addition, all technical starch breakdown products such as glucose syrup or dextrins, e.g. maltodextrins, come into consideration as starting material for the group Z. Preferably, this gives for the variable Z a polyhydroxyalkyl group of the formula —CH₂—(CHOH)₄—CH₂OH derived from aldohexoses. Particularly preferably, this can be a group of glucose, in particular D-glucose.

As base in aqueous solution, in the method according to the invention, alkoxides and/or alkali metal hydroxides can be used. Preferably, in particular C₁-C₄ alkoxides with alkali metal counterions or alkali metal hydroxides such as, for example, LiOH, NaOH, KOH or mixtures thereof can be used. In the context of the method, the concentration of the bases in the aqueous solution can be greater than or equal to 0.05 N and less than or equal to 5 N, preferably greater than or equal to 0.05 N and less than or equal to 4 N, and further preferably greater than or equal to 0.05 N and less than or equal to 3 N. Within this concentration range, high reaction rates of the N-alkylglucamine may be obtained.

In step c), at least two carboxylic acid alkyl esters having different C₆-C₂₂ chain lengths are used. The carboxylic acid alkyl esters conform to the formula R—CO—O—R², wherein R² is a C₁ to C₄ alkyl group such as, for example, methyl, ethyl, n-propyl, isopropyl, n-butyl, isobutyl, sec-butyl, tert-butyl. Preferably, as alkyl groups of the carboxylic acid alkyl esters, methyl and ethyl esters can be used. As carboxylic acid alkyl esters, according to the invention, also mono-, di- or triglycerides having different C₆-C₂₂ chain lengths may be used. In particular, partially crystalline glucamide compositions may also be obtained by the method according to the invention from triglyceride oils such as, for example, coconut oil or palm kernel oil.

The alkyl group R of the carboxylic acid segment can represent a C₅-C₂₁ saturated or monounsaturated/polyunsaturated alkyl group. Preferably, the carboxylic acid ester can have been formed by esterification from a long-chain, naturally occurring fatty acid such as, for example, lauric, myristic, palmitic and/or stearic acid. In addition, in particular a mixture of at least two carboxylic acid alkyl esters can be present when the fraction of each carboxylic acid alkyl ester makes up at least greater than or equal to 5 mol %, preferably greater than or equal to 10 mol %, and further preferably greater than or equal to 15 mol % of the carboxylic acid alkyl ester total amount. Despite this mixture of different carbon chain lengths, partially crystalline glucamide structures can be formed.

In addition, in an additional embodiment of the method, the base of steps a) can be sodium hydroxide solution. Sodium hydroxide solution has proved to be particularly suitable in the context of the process procedure for efficient conditioning of the N-alkylglucamine. In the context of the method, the NaOH concentration in the reaction solution can be greater than or equal to 0.05 N and less than or equal to 5 N, preferably greater than or equal to 0.1 N and less than or equal to 3 N, and in addition preferably greater than or equal to 0.2 N and less than or equal to 2 N. This can lead to highly selective reactions with only a very low fraction of unreacted N-alkylglucamine.

According to a further embodiment of the method according to the invention, the water content of the dried reaction product after step b) can be greater than or equal to 0.05% by weight and less than or equal to 0.4% by weight. Surprisingly, it has been found that complete drying of the reaction product of step a) can be disadvantageous for the further method procedure. Small fractions of water after the drying appear able to accelerate the reaction. In addition, the residual water content also appears to have effects on the regioselectivity of the subsequent reaction. For this reason, further preferably, a residual water content of the reaction product after step b) of greater than or equal to 0.1% by weight and less than or equal to 0.4% by weight and further preferably greater than or equal to 0.15% by weight and less than or equal to 0.35% by weight can be present. Larger amounts of water can, in contrast, have disadvantageous effects on the formation of the partially crystalline structure. The residual water content in this stage can be effected by methods known to those skilled in the art. Expediently, the detection can proceed by a Karl-Fischer titration of the dried product.

In a further characteristic of the method according to the invention, the carboxylic acid alkyl esters can be initially charged in step c) in a solvent selected from the group consisting of mono-, di- and/or trihydric alcohols. Carrying out the reaction in step c) in the presence of a monohydric or polyhydric alcohol can contribute to a reduction of the viscosity of the reaction solution and thereby to a faster and more complete reaction. Possibly, by the use of the alcohols, a more uniform temperature procedure may also be achieved. This can have an effect on the completeness of the reagent conversion, and also on the byproduct profile. Preferably, step c) can proceed in diols, wherein propylene glycol can preferably be used. Adequate concentration ranges for the solvents can result in ranges between greater than or equal to 5% by weight and less than or equal to 70% by weight, preferably between greater than or equal to 10% by weight and less than or equal to 50% by weight, and further preferably between greater than or equal to 10% by weight and less than or equal to 40% by weight.

In addition, in a further aspect of the method according to the invention, the method step e) can be carried out in a temperature range of greater than or equal to 50° C. and less than or equal to 175° C. Reacting the composition to completion can preferably proceed under the action of heat. An efficient method procedure has resulted in this case in the temperature range stated above. Equally good results, however, may also be obtained in that the mixture is reacted to completion in a temperature range of greater than or equal to 50° C. and less than or equal to 150° C., preferably greater than or equal to 70° C. and less than or equal to 130° C., and further preferably greater than or equal to 85° C. and less than or equal to 110° C. Within this temperature range, an efficient and complete reaction can result with very low byproduct concentrations.

Expediently, the method step e) is carried out under reduced pressure. By means of the reduced pressure in this case, volatile reaction components can be taken off. To obtain a byproduct fraction as small as possible, the pressure can be controlled to greater than or equal to 10 mbar and less than or equal to 100 mbar, and further preferably to greater than or equal to 25 mbar and less than or equal to 50 mbar. For example low-boiling alcohols may thereby be removed from the reaction mixture and thus shift the reaction equilibrium in the direction of the products.

In a further embodiment of the method according to the invention, the reaction product of step e) can be adjusted to an N-alkyl-N-acylglucamine content of greater than or equal to 2.5% by weight and less than or equal to 50% by weight in an additional method step by addition of at least one protic solvent selected from the group consisting of water, mono-, di- and/or trihydric alcohols or mixtures thereof. Surprisingly, the compositions according to the invention may be diluted, while retaining the at least partially crystalline structure thereof. This can expediently proceed by adding the abovementioned solvents. Without wishing to be bound by theory, this is very probably possible since the fraction of unwanted byproducts is extremely low owing to the chosen method procedure. For this reason, the interactions between the N-alkyl-N-acylglucamines can be particularly high, in such a manner that addition/incorporation even of further solvent molecules can proceed without destroying the partially crystalline structures.

The invention further relates to the use of a partially crystalline N-alkyl-N-acylglucamine composition in cleaning, cosmetics, dermatological and/or pharmaceutical applications. It has advantageously appeared that the partially crystalline compositions, on account of the property profile thereof, are particularly suitable for the use in the abovementioned fields. The compositions are highly dermatologically acceptable, demonstrate good foaming and cleaning properties and are biodegradable. Without wishing to be bound by theory, the high dermatological acceptability can also result from the formation of the partially crystalline structures. Possibly, the partially crystalline arrangement of the glucamides may decrease the diffusion into and the interaction with lower skin structures in such a manner that the barrier action of the skin is retained to a high extent. This is in contrast to surfactant micelles or individual surfactant molecules of other amphiphiles which can penetrate unhindered into lower dermal layers, for example through skin pores or the lipid layers.

In particular, within a further use aspect, the cleaning application can be a rinse-off personal care application. In particular, the compositions according to the invention can be useful as cleaning agents on account of their viscosity and foaming properties. This is due in particular to their dermatological compatibility with skin surfaces or else with hair. If, in addition, highly effective cleaning systems are demanded which are to work effectively within a very short time period, these compositions can be useful precisely in the field of “rinse-off” products that can be washed off after the application.

With respect to further advantages and features of the use described above, reference is made hereby explicitly to the explanations in connection with the composition according to the invention and also the method according to the invention. Also features according to the invention and advantages of the method according to the invention are also intended to be applicable to the composition according to the invention and the use according to the invention and are considered as disclosed, and vice versa. Also all combinations of at least two of the features disclosed in the description and/or the claims come under the invention.

The invention will be explained in more detail hereinafter with reference to exemplary embodiments.

For the powder X-ray diffraction (PXRD), the sample preparations were made on a rotating “zero background” sample carrier made of silicon. The measurements were made in reflection at room temperature (20-25° C.) using a Bruker D8 Advance-Diffractometer (Cu—Kα1=1.54059 Å; Johansson primary beam monochromator, position-sensitive detector). The measuring time was two hours in each case. To evaluate the data, the EVA program version 14.0 from Bruker-AXS was used. Determination of the peak intensities in counts/sec background correction using the program settings threshold=1 and also curvature=1 was performed. Normalization took place with in each case the signal maximum (100%) of the highest intensity (counts/sec) in the 2Theta range 3-40°.

Significant reflections are present in the meaning of the invention when the relation between maximally observed intensity in the angle range considered is greater than or equal to the fraction given at the highest measured intensity in the 2Theta range between 3° and 40°. The following relations result:

| 2Theta range | % fraction of the intensity in relation |
|---|---|
| considered | to intensity maximum (3°-40°) |
| ≧15.5° to ≦17.4° | ≧10% |
| ≧13.5° to ≦15.1° | ≧5% |
| ≧11.6° to ≦12.6° | ≧7% |
| ≧7.5° to ≦8.5° | ≧20% |
| ≧6.2° to ≦7.5° | ≧5% |
| ≧5.6° to ≦6.5° | ≧3% |

PXRD spectra were recorded using the above-specified method on numerous different N-methylglucamides which had been obtained by means of the method according to the invention.

In the drawings:

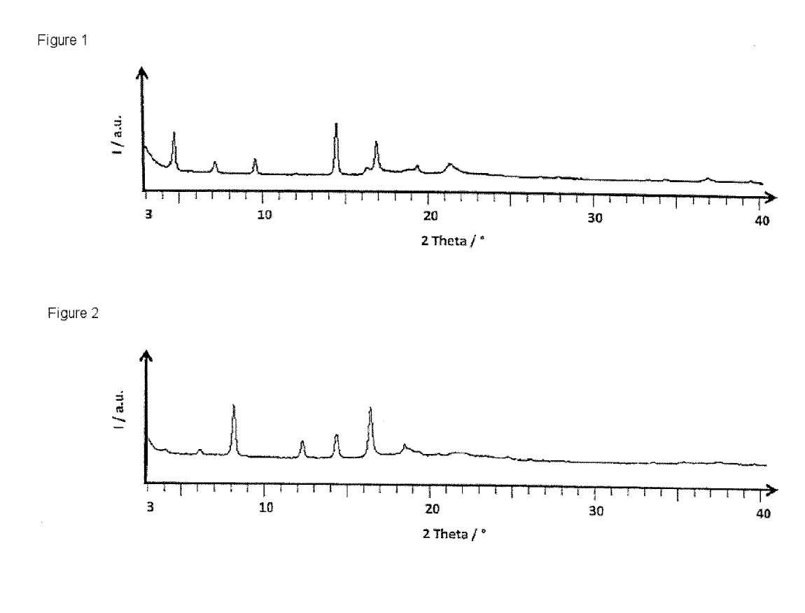

FIG. 1 shows the PXRD spectrum of a composition according to the invention having a C₈/C₁₀ glucamide chain section

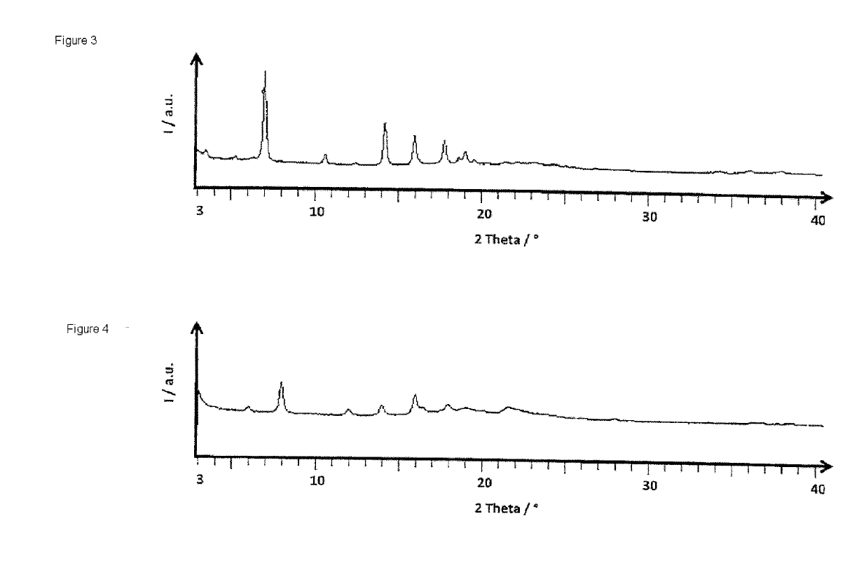

FIG. 2 shows the PXRD spectrum of a composition according to the invention having a C₁₂/C₁₄ glucamide chain section

FIG. 3 shows the PXRD spectrum of a composition according to the invention having a C₁₆/C₁₈ glucamide chain section

FIG. 4 shows the PXRD spectrum of a composition according to the invention having a B12/C_{14/}C_{16/}C₁₈′ glucamide chain section

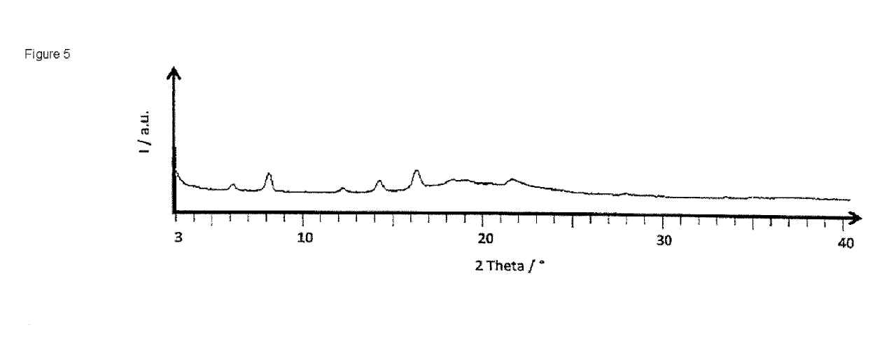

FIG. 5 shows the PXRD spectrum of a composition according to the invention having a C₈/C₁₀/C₁₂/C₁₄/C₁₆/C₁₈/C₁₈′ glucamide chain section

FIG. 1 shows an X-ray powder diffraction diffractogram recorded in Bragg-Brentano geometry of a partially crystalline N-alkyl-N-acylglucamine composition according to the invention in the range between 3° and 40° (2Theta). The partially crystalline structures may be clearly recognized with reference to the reflections in the 2Theta ranges ≧13.5° to ≦15.1° and ≧15.5° to ≦17.4°. The sample is a C₈/C₁₀ N-methylglucamide chain section, wherein 40 mol % of the glucamides bear a C₈ chain group and 60 mol % of the glucamides bear a Cio chain group. The fraction of glucamides in the sample is approximately 90% by weight. The remainder of the sample (approximately 10% by weight) comprises propylene glycol. Traces of water may also be present.

FIG. 2 shows an X-ray powder diffraction diffractogram recorded in Bragg-Brentano geometry of a partially crystalline N-alkyl-N-acylglucamine composition according to the invention in the range between 3° and 40° (2Theta). The partially crystalline structures may be clearly recognized with reference to the reflections in the 2Theta ranges ≦13.5° to ≦15.1° and ≧15.5° to ≦17.4°. The sample is a C₁₂/C₁₄ N-methylglucamide chain section, wherein 75 mol % of the glucamides bear a C₁₂ chain group and 25 mol % of the glucamides bear a C₁₄ chain group.

In addition, the diffractogram further shows a significant X-ray reflection in the 2Theta range of ≧7.5° to ≦8.5°. The fraction of glucamides of the sample is approximately 90% by weight. The remainder of the sample (approximately 10% by weight) comprises propylene glycol. Traces of water may also be present.

FIG. 3 shows an X-ray powder diffraction diffractogram recorded in Bragg-Brentano geometry of a partially crystalline N-alkyl-N-acylglucamine composition according to the invention in the range between 3° and 40° (2Theta). The partially crystalline structures may be clearly recognized with reference to the reflections in the 2Theta ranges ≧13.5° to ≦15.1° and ≧15.5° to ≦17.4°. In addition, the diffractogram further shows a significant

X-ray reflection in a 2Theta range of ≧6.2° to ≦7.5°. The sample is a C₁₆/C₁₈ N-methylglucamide chain section, wherein 60 mol % of the glucamides bear a C₁₆ chain group and 40 mol % of the glucamides bear a C₁₈ chain group. The fraction of glucamides of the sample is approximately 80% by weight. The remainder of the sample (approximately 20% by weight) comprises propylene glycol. Traces of water may also be present.

FIG. 4 shows an X-ray powder diffraction diffractogram recorded in Bragg-Brentano geometry of a partially crystalline N-alkyl-N-acylglucamine composition according to the invention in the range between 3° and 40° (2Theta). The partially crystalline structures may be clearly recognized with reference to the reflections in the 2Theta ranges ≧13.5° to ≦15.1° and ≧15.5° to ≦17.4°. The sample is a C₁₂/C₁₄/C₁₆/C₁₈/C₁₈′ N-methylglucamide chain section, wherein 62 mol % of the glucamides bear a C₁₂ chain group, 24 mol % of the glucamides bear a C₁₄ chain group, 5 mol % of the glucamides bear a C₁₆ chain group, 1 mol % of the glucamides bear a C₁₈ chain group and 8 mol % of the glucamides bear a C₁₈′ chain group. The designation C₁₈′ chain group means that the C₁₈′ chain group bears at least one unsaturated double bond. Surprisingly, despite the double bond in some of the chains, a partially crystalline structure is found. This is surprising in particular since double bonds, owing to their restricted rotation, can usually make formation of crystalline structures markedly more difficult. The fraction of glucamides in the sample is approximately 88% by weight. The remainder of the sample (approximately 12% by weight) comprises propylene glycol. Traces of water may also be present.

FIG. 5 shows an X-ray powder diffraction diffractogram recorded in Bragg-Brentano geometry of a partially crystalline N-alkyl-N-acylglucamine composition according to the invention in the range between 3° and 40° (2Theta). The partially crystalline structures may be clearly recognized with reference to the reflections in the 2Theta ranges ≧13.5° to ≦15.1° and ≧15.5° to ≦17.4°. The sample is a C₈/C₁₀/C₁₂/C₁₄/C_{16/}C₁₈/C₁₈′ N-methylglucamide chain section, wherein 8 mol % of the glucamides bear a C₈ chain group, 6 mol % of the glucamides bear a Cio chain group, 48 mol % of the glucamides bear a C₁₂ chain group, 18 mol % of the glucamides bear a C₁₄ chain group, 9 mol % of the glucamides bear a C₁₈ chain group, 3 mol % of the glucamides bear a C₁₈ chain group and 8 mol % of the glucamides bear a C₁₈′ chain group. The designation C₁₈′ chain group means that the C₁₈′ chain group bears at least one unsaturated double bond. The fraction of glucamides in the sample is approximately 83% by weight. The remainder of the sample (approximately 17% by weight) comprises propylene glycol and glycerol. Traces of water may also be present.

Synthesis of the N-alkyl-N-acylglucamines:

In a first step, an N-alkylglucamine, for example N-methylglucamine (NMG), is added together with concentrated aqueous sodium hydroxide solution. The concentration of sodium hydroxide in the composition can here, for example, be between 0.5 and 1.5% by weight. The precursor is allowed to react to completion for at least 2 h and the precursor is then dried with supply of heat. Optionally, the drying can also be promoted by applying a partial vacuum. Preferably, the precursor is not completely dewatered here.

In a second method step, at least two carboxylic acid alkyl esters having different carbon chain lengths, or mono-, di- or triglycerides having different carbon chain lengths are initially charged. The carboxylic acid alkyl esters can also be initially charged in a solvent such as, for example, propylene glycol. The carboxylic acid alkyl esters or the carboxylic acid alkyl ester/solvent are heated to a temperature of between 50° C. and below or equal to 150° C. For example, the composition can be heated to 100° C. Then, the dried precursor from step 1) is added in portions to this heated mixture. Optionally, highly volatile reaction products can be removed from the reaction mixture by a partial vacuum, for example in a pressure range between 25 mbar-50 mbar. After addition of the entire precursor, the mixture is allowed to react to completion for a further two hours and the N-alkylglucamides are obtained. Depending on the method procedure, the N-alkylglucamide composition can contain a solvent, for example propylene glycol, and, when triglycerides are used, possibly glycerol.

In a further method step, the composition can be further diluted by water or further organic solvents such as alcohols, with input of stirrer energy.

1. A partially crystalline N-alkyl-N-acylglucamine composition comprising at least two different acylglucamines having acyl groups selected from the group consisting of saturated or unsaturated C₆-C₂₂ acyls, wherein the composition at room temperature has a partially crystalline structure with at least one significant X-ray reflection in each of the 2Theta ranges ≧13.5° to ≦15.1° and ≧15.5° to ≦17.4°, wherein the X-ray reflections are obtained by a powder diffraction diffractogram in Bragg-Brentano geometry.

2. The partially crystalline N-alkyl-N-acylglucamine composition as claimed in claim 1, wherein the partially crystalline N-alkyl-N-acylglucamine composition at room temperature has at least one further significant X-ray reflection in a 2Theta range from ≧7.5° to ≦8.5°.

3. The partially crystalline N-alkyl-N-acylglucamine composition as claimed in claim 1, wherein the partially crystalline N-alkyl-N-acylglucamine composition at room temperature has at least one further significant X-ray reflection in a 2Theta range from ≧6.2° to ≦7.5°.

4. The partially crystalline N-alkyl-N-acylglucamine composition as claimed in claim 1, wherein the partially crystalline composition comprises C₁₂ N-methyl-N-acylglucamine and the C₁₂ acylglucamine fraction, based on the total N-methyl-N-acylglucamine content, is greater than or equal to 25 mol % and less than or equal to 95 mol %.

5. The partially crystalline N-alkyl-N-acylglucamine composition as claimed in claim 1, wherein the partially crystalline N-alkyl-N-acylglucamine composition additionally comprises a solvent selected from the group consisting of mono-, di- or trihydric alcohols.

6. The partially crystalline N-alkyl-N-acylglucamine composition as claimed in claim 1, wherein the concentration of the N-alkyl-N-acylglucamines in the partially crystalline N-alkyl-N-acylglucamine composition is greater than or equal to 5% by weight and less than or equal to 65% by weight.

7. The partially crystalline N-alkyl-N-acylglucamine composition as claimed in claim 1, wherein the fraction of at least one N-alkyl-N-acylglucamine in the partially crystalline N-alkyl-N-acylglucamine composition is greater than or equal to 40 mol %, and less than or equal to 95 mol %, based on the total N-alkyl-N-acylglucamine content.

8. A method for preparing a partially crystalline N-alkyl-N-acylglucamine composition comprising the steps:
a) bringing the N-alkylglucamine to reaction with a base in aqueous solution to form a reaction product,
b) drying the reaction product of step a) to form a dried reaction product,
c) initially charging at least two carboxylic acid alkyl esters having a different C₆-C₂₂ chain length,
d) adding in portions the dried reaction product of obtained under b) to the mixture of c) and
e) reacting to completion the composition of d) under reduced pressure.

9. The method as claimed in claim 8, wherein the base of steps a) is sodium hydroxide solution.

10. The method as claimed in claim 8, wherein the water content of the dried reaction product after step b) is greater than or equal to 0.05% by weight and less than or equal to 0.4% by weight.

11. The method as claimed in claim 8, wherein the carboxylic acid alkyl esters are initially charged in step c) in a solvent selected from the group consisting of mono-, di- and/or trihydric alcohols.

12. The method as claimed in claim 8, wherein the method step e) is carried out in a temperature range of greater than or equal to 50° C. and less than or equal to 175° C.

13. The method as claimed in claim 8, wherein the reaction product of step e) is adjusted to an N-alkyl-N-acylglucamine content of greater than or equal to 2.5% by weight and less than or equal to 50% by weight in an additional method step by addition of at least one protic solvent selected from the group consisting of water, mono-, di- and/or trihydric alcohols or mixtures thereof.

14. A cleaning, cosmetics, dermatological and/or pharmaceutical composition comprising a partially crystalline N-alkyl-N-acylglucamine composition as claimed in claim 1.

15. The cleaning, cosmetics, dermatological and/or pharmaceutical composition as claimed in claim 14, wherein the cleaning application is a rinse-off personal care application.
